(19) United States
(12) Patent Application Publication
(10) Pub. No.: US 2007/0266449 A1
(43) Pub. Date: Nov. 15, 2007

(54) GENERATION OF ANIMAL MODELS

(76) Inventor: Robert A. Zivin, Cranbury, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/748,046

(22) Filed: May 14, 2007

Related U.S. Application Data
(60) Provisional application No. 60/799,807, filed on May 12, 2006.

(51) Int. Cl. A01K 67/027

(52) U.S. Cl. 800/9

(57) ABSTRACT

The present invention is directed to the generation of animal models of human disease via the targeted alteration of the nucleic acid sequences in genes encoding drug target proteins. The animal models of human disease may be used for the evaluation of candidate drug molecules.

### FIELD OF THE INVENTION

This invention relates to methods to generate animal models of human disease. In particular, this invention is directed to the generation of animal models of human disease by the targeted alteration of the genes encoding drug targets. The animal models of human disease may be used for the evaluation of candidate drug molecules.

### BACKGROUND

The systematic screening of natural and synthetic compounds to find potential drug candidates has been a cornerstone of pharmaceutical research for many decades. A common approach is to first screen a library of candidate drug molecules in assays against a drug target. The drug target may be, for example, a recombinant protein in solution, or the drug target may be a protein expressed in a cell line in vitro.

For example, Yamano et al (Biochem. Pharmacol. 2005) utilized in vitro receptor binding assays using purified cell membranes containing human adenosine A3 receptors to identify a class of compounds that are highly selective antagonists and that bind the human adenosine A3 receptor with high affinity.

Thurmond et al (JPET 308: 268-276, 2004) utilized in vitro enzyme kinetic assays using purified recombinant human cathepsin S to identify classes of compounds that are highly selective antagonists for human cathepsin S.

Mammalian cells may be genetically engineered to over-express a target protein of interest. Engineered cell lines may be used in vitro in a functional assay to screen for potential drug candidates. Such functional assays may include, for example, recording changes in intracellular calcium, or changes in membrane potential. Grant et al (JPET 300: 9-17, 2002) report the use of a cell line over-expressing the human vanilloid receptor 1 to determine the pharmacological properties of human vanilloid receptor 1 antagonists.

However, in vitro assays may not always reflect how a candidate drug molecule interacts with a drug target protein within the complex milieu of an intact cell or organism. Pharmaceutical research may therefore strongly benefit from the availability of suitable animal models of human disease. That said, differences exist between humans and animals. For example, the human and animal homologues of a specific drug target protein may display structural differences at the molecular level, thereby resulting in species-specific pharmacological properties. These species differences present considerable problems in the pharmacological evaluation of candidate drug molecules. The differences may be, for example, differences in the amino acid sequence at, or around the binding site of the candidate drug molecule. The differences may be one, or a combination of, a deletion, a substitution or an addition of a single, or multiple amino acids. Such differences can negatively impact the relevance of a drug screening assay.

For example, Yamano et al states supra: “All of the human adenosine A3 receptor antagonists show extremely low binding affinity for the rodent adenosine A3 receptor. The large species differences between rodent and human adenosine A3 receptors and the lack of highly potent antagonists for the rodent adenosine A3 receptor are currently serious drawbacks in the further pharmacological evaluation of adenosine A3 receptor antagonists”.

Thurmond et al report the discovery of a novel class of immunosuppressive compounds that are highly potent, nonpeptidic, noncovalent inhibitors of human cathepsin S, but they are much less active against the mouse, dog, monkey, and bovine enzymes. In the case of murine cathepsin S, the amino acid sequence of the drug binding site differs from the human sequence by a single amino acid, yet that is sufficient to significantly decrease the binding affinity of candidate drug molecules.

As another example, the pharmacological and/or toxicological side effects of a candidate drug molecule may be assessed by determining the changes in the levels of expression of members of the cytochrome P450-dependent monooxygenase system (CYP450). These enzymes are expressed in the liver. However, results using CYP450 assay systems are often inconsistent, due to species differences in the expression of the cytochrome P450-dependent monooxygenase enzymes.

Replacing the organ of interest with the human organ may humanize animal models. Alternatively, an organ derived from an animal may be replaced or repopulated in whole or in part with human cells. For example, Katoh et al report the use of chimeric mice to determine the pharmacological and/or toxicological side effects of drug candidates by measuring changes in expression levels of members of the cytochrome P450-dependent monooxygenase system. In these studies, the chimeric mice were generated by partially repopulating the mouse liver with human hepatocytes. While these models are useful to a point, in these studies, the recipient animals died when the replacement index of the human hepatocytes exceeded 50%.

An alternative approach has been to generate transgenic animals, where the animal gene encoding the drug target protein has been deleted or silenced, and the human gene, encoding the human drug target protein has been introduced. In one example, Yamano et al generated mice in which the murine adenosine A3 receptor gene was replaced by its human counterpart, in order to evaluate the pharmacological effects of human adenosine A3 receptor antagonists in mouse models.

Most of the current methods used to introduce the respective human gene into a transgenic animal model rely on the random integration of the transgene into the genome of a recipient cell. This random integration can disrupt genes at the integration site or cause the expression of genes near the integration site to become misregulated.

Genetic manipulations that allow the precise replacement of one sequence with another can be accomplished through homologous recombination. Unfortunately, homologous recombination is a rare event in mammalian cells, while random integration is much more frequent. The creation of a targeted DNA double-strand break, however, has been shown to stimulate homologous recombination several hundred-fold by activating the cellular machinery responsible for homology-directed repair. Double-stranded breaks may be introduced at specific regions of DNA by the use of targeted nucleases. For example, WO 03/087341 disclose zinc finger nucleases that comprise an engineered DNA binding domain of a Cys2His2 zinc finger transcription factor coupled to an exonuclease, such as, for example, the nonspecific DNA cleavage domain of the Type IIS restriction enzyme, FokI.

WO 03/087341 discloses methods where zinc finger nucleases are utilized to disrupt a gene in a somatic cell, wherein that gene is over-expressing a product and/or expressing a product that is deleterious to the cell or organism. WO 03/087341 also discloses methods where zinc finger nucleases are utilized to enhance expression of a particular gene by the insertion of a control element into a somatic cell. In addition, WO 03/087341 discloses methods where zinc finger nucleases are utilized to insert donor DNA s encoding a gene product that, when constitutively expressed, has a therapeutic effect. WO 03/087341 states “An example of this embodiment would be to insert such DNA constructs into an individual suffering from diabetes in order to effect insertion of an active promoter and donor DNA encoding the insulin gene in a population of pancreatic cells”.

WO2004037977 discloses methods to ameliorate a genetic disorder in a subject. In addition, WO2004037977 discloses methods to confer a desirable genotype on a subject or cell. WO2004037977 also discloses methods to increase the production or activity of a beneficial polypeptide in a subject or cell”. WO2004037977 states, “the subject methods may be used to introduce a transgene for expression in the cell. For example, a genetic disease caused by a decrease in the level of a necessary gene product may be treated or ameliorated by providing a transgene expressing the needed gene product.”

The methods described above result in either the inactivation of an entire gene, the replacement of an entire gene, or the over expression of an entire gene. Furthermore, the gene that is inserted into the recipient cell DNA using the methods disclosed above is generally no longer under the physiological control of the recipient cell DNA. Instead, the gene is more often under the control of regulatory elements introduced with the inserted DNA.

The present invention takes advantage of engineered zinc finger nucleases to target specific regions within a gene encoding the animal homologue of a specific protein that differs from the human gene. The zinc finger nuclease introduces a DNA double-stranded break at these specific regions, which enable the introduction of exogenous nucleotide sequences by homologous recombination. The animal gene is not entirely replaced: rather, only specific regions of the animal gene are altered. Therefore, the altered gene remains under the appropriate physiological regulation.

### SUMMARY

The present invention is directed to the generation of animal models of human disease for the identification and characterization of candidate drug molecules. The animal models generated by the methods of the present invention express a protein that is altered at least one specific region.

The gene encoding the protein is identified in the animal, and is compared to the corresponding human gene that encodes the protein. The region or regions in the animal gene that differ from the human gene are altered by homologous recombination.

The pharmacological properties of the animal protein are altered to resemble the human homologue of the protein.

In one embodiment, the animal protein is an animal drug target protein. In an alternate embodiment, the animal protein is a protein that interacts with an animal drug target protein.

A single animal cell may be treated according to the methods of the present invention. Alternatively, a population of animal cells may be treated. The treated cell or cells may be expanded in vitro for use in in vitro drug screening assays.

A single cell may be treated according to the methods of the present invention and the treated cell may be used to generate a transgenic animal. The resulting transgenic animal may be used as a model for human disease directly, or, alternatively, as a source of tissue expressing the human protein.

### BRIEF DESCRIPTION OF THE DRAWINGS

FIG. 1: Schematic representation of zinc finger nucleases. Panel A shows a schematic representation of a zinc finger nuclease suitable for use in the present invention. Panel B shows a schematic representation of a zinc finger nuclease suitable for use in the present invention bound to its target locus. Panel C shows an outline of the method of the present invention.

FIG. 2: Alignment of the human, monkey, mouse and dog amino acid sequences of cathepsin S.

### DETAILED DESCRIPTION

For clarity of disclosure, and not by way of limitation, the detailed description of the invention is divided into the following subsections that describe or illustrate certain features, embodiments or applications of the present invention.

For the purposes of the present invention, the term “zinc finger nuclease” refers to a chimeric protein molecule comprising at least one zinc finger DNA binding domain effectively linked to at least one nuclease capable of cleaving DNA. A zinc finger nuclease of the present invention is capable of directing targeted genetic recombination or targeted mutation in a host cell by causing a double-stranded break at a target locus. A zinc finger nuclease of the present invention includes a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the chimeric protein molecule and the DNA-cleavage domain is located at the C-terminus of the molecule.

The term “chimeric protein” is used to describe a protein that has been expressed from a DNA molecule that has been created by joining two or more DNA fragments. The DNA fragments may be from the same species, or they may be from a different species. The DNA fragments may be from the same or a different gene.

The term “nuclease” refers to an enzyme that breaks down the chemical bonds between nucleic acids in a nucleotide chain. The term “DNA cleavage domain” refers to the region in a zinc finger nuclease that is capable of breaking down the chemical bonds between nucleic acids in a nucleotide chain. Examples of proteins containing cleavage domains include restriction enzymes, topoisomerases, recombinases, integrases and DNAses.

The term “zinc finger binding domain” refers to the region in a zinc finger nuclease that is capable of binding to a target locus in a gene.

The term “locus” refers the position in a chromosome of a particular gene, or allele, or nucleotide sequence. The term “target locus” refers to the position in a chromosome of a particular gene, or allele, or nucleotide sequence where a zinc finger nuclease of the present invention binds. The target locus for a zinc finger nuclease of the present invention is within at least 500 base pairs, and alternatively within at least 200, or alternatively at least 100 base pairs of a region of an animal gene that differs from that of the corresponding human gene.

The terms “differs”, or “different”, or “differences” are used to indicate a region of an animal gene or protein that is unlike or distinct in nature, form, or characteristics from that of the human homologue of the gene or protein.

For the purposes of the present invention, the term “sequence” means any contiguous region of nucleic acid bases or amino acid residues, and may or may not refer to a sequence that encodes or denotes a gene or a protein. For the purposes of the present invention, the term “adjacent” is used to indicate two elements that are next to one another without implying actual fusion of the two elements. Additionally, for the purposes of the present invention, “flanking” is used to indicate that the same, similar, or related sequences exist on either side of a given sequence. Segments described as “flanking” are not necessarily directly fused to the segment they flank, as there can be intervening, non-specified DNA between a given sequence and its flanking sequences.

These and other terms used to describe relative position are used according to normal accepted usage in the field of genetics.

For the purposes of the present invention, the term “gene” refers to a nucleic acid sequence that includes the translated sequences that encode a protein (“exons”), the untranslated intervening sequences (“introns”), the 5′ and 3′ untranslated region and any associated regulatory elements.

For the purposes of the present invention, the term “recombination,” is used to indicate the process by which DNA at a given locus is altered as a consequence of an interaction with other DNA. For the purposes of the present invention, the term “homologous recombination” is used to indicate recombination occurring as a consequence of interaction between segments of DNA that are homologous, or identical.

As used herein, the term “donor DNA” or “donor DNA construct” refers to the entire set of DNA segments to be introduced into the host cell or organism as a functional group. The term “segments” as used herein refers to sequences of nucleic acids. The term “donor DNA” as used herein refers to a DNA segment with sufficient homology to the region of the target locus to allow participation in homologous recombination at the location of the region of difference.

A drug is any substance that is intended for use in the diagnosis, cure, mitigation, treatment, or prevention of disease, or affects the structure or function of the body. Drug molecules vary in size, the molecular weight of most drugs falls within the range of about 100 daltons to about 1,000 daltons. Drug substances are often small or relatively small organic molecules. Peptides and proteins, such as, for example antibodies are also considered to be drugs within the context of the present invention, irrespective of whether they are administered from outside the body or their release is stimulated endogenously. A candidate drug molecule is any substance that may be obtained and tested for its potential to become a drug. A candidate drug molecule of the present invention may be obtained from its natural source, or it may be produced using molecular biology techniques or it may be produced by chemical synthesis.

The term “drug target protein” refers to a protein to which a drug molecule selectively binds.

The present invention is directed to an improved method to generate an animal model of disease. The improvement comprises the targeted alteration of sequences within genes encoding proteins by homologous recombination. The present invention does not replace the entire gene in the animal: rather, only specific regions are altered, leaving the gene under the appropriate physiological control. The specific regions are sequences in the animal gene that have been found to differ from that of the human gene homologue.

In the present invention, the targeted alteration of sequences within genes by homologous recombination is facilitated by the introduction of DNA double-stranded breaks in the gene. This is achieved by the use of zinc finger nucleases, or other engineered enzymes comprising a DNA binding domain and a DNA cleavage domain. These proteins are capable of binding to DNA at a specific locus and introducing double-stranded breaks into the DNA of the gene, allowing homologous recombination to occur.

In one aspect of the present invention, donor DNA is inserted into a specific locus in the gene or genes encoding a protein in animal cells by a method comprising:
1) identifying at least one region in an animal protein that differs from a corresponding human protein homologue;
2) determining the nucleotide sequences that encode the region of difference in the human and animal genes;
3) identifying a target locus for a zinc finger nuclease within the identified region of difference in the animal gene;
4) determining the nucleotide sequences that flank the region of difference in the animal gene;
5) generating donor DNA, the donor DNA comprising an oligonuelcotide encoding the human region of difference, flanked by the sequences determined in step 4;
6) generating a zinc finger nuclease that is capable of binding to the target locus;
7) introducing the donor DNA and the zinc finger nuclease into animal cells, thereby inducing double-stranded breaks in the DNA of the animal cells;
8) allowing homologous recombination to occur between the animal DNA and the donor DNA, and;
9) selecting the animal cells where homologous recombination has occurred.

In one embodiment, the animal protein is an animal drug target protein. In an alternate embodiment, the animal protein is a protein that interacts with an animal drug target protein.

The present invention is used when the amino acid sequence of the animal protein differs from the amino acid sequence of the human protein homologue by at least one amino acid. The difference may be in the form of a deletion, an insertion, a substitution of an amino acid, or a combination of the above. Preferably, the present invention is used when the differences in amino acid sequence correspond to the region of the human protein that contains the site where the candidate drug molecules bind. Alternatively, the differences in amino acid sequences may correspond to a region of the protein that contains the site where other proteins or molecules bind that impact the effect of the drug on the human cell.

The methods of the present invention result in the alteration of the amino acid sequence of an animal protein via alteration of the gene encoding the animal protein to a form more equivalent to the corresponding human protein using homologous recombination. The alteration of the gene sequence is at a specific region, corresponding to the region that encodes the specific amino acid sequence that has been determined to differ.

As described above, one challenge in developing animal models for human disease is that a human protein and its comparable animal protein will differ in their interaction with a candidate small molecule. These differences can impact what drug is developed, how the drug is developed, the formulations developed and the initial dosage forms. Animal models support the development of drugs for that animal in many instances. Alteration of the animal protein by the methods of the present invention effectively humanize the protein and enable the animal protein to bind or interact with candidate drug molecules in a way that more effectively mimics the interaction of the candidate drug with a human protein.

For example, the animal drug target protein may bind or interact with candidate drugs with a lower affinity than the human drug target protein. Alteration of the animal drug target protein by the methods of the present invention may increase the affinity of the binding or interaction with the candidate drug molecules.

In another example, the drug target protein interacts with a second protein. This interaction may be required for the binding of the candidate drug molecule to the drug target protein. Alternatively, this interaction may cause an adverse drug reaction. These interactions may vary between animals and humans. The present invention provides methods by which the interaction of the animal drug target protein with a second protein resembles the interaction of the human drug target protein with a second protein.

### Identification of the Regions of Difference in Proteins and Determination of a Target Locus

The present invention requires the identification of regions of proteins that interact with, or bind candidate drug molecules. Regions of proteins that bind to and interact with other proteins may also have to be identified. The amino acid sequence of the region or regions, together with the three-dimensional structure of these regions and how they differ between the animal and the human are useful in determining which regions to alter by the methods of the present invention.

A variety of techniques may be employed to identify the regions of the protein that interact with or bind to the candidate drug molecules. These include, for example, amide deuterium exchange-mass spectrometry, according to the methods disclosed in, for example, Woods and Hamuro, J. Cell Biochem. 84: 89-98, 2002.

In this technique, the precise locations of attached deuterium label within the known amino acid sequence of the protein may then be determined by proteolysis of the labeled protein into peptide fragments. The peptide fragments are then subjected to rapid high performance liquid chromatography (HPLC) separation, and directly analyzed by electrospray-ion trap or time of flight (TOF) mass spectrometry performed under conditions adapted to amide exchange studies. Peptides that contain deuterium, indicative of prior functional labeling, are identified. Additional proteases may be employed to accomplish progressive proteolysis, which may increase the resolution of the peptide mapping.

The candidate drug molecule-binding site may be determined in both the animal and human protein homologue according to the method outlined above. The amino acid sequences of the binding sites of both proteins may be compared. If the amino acid sequences differ by at least one amino acid, the region may be identified as a region of difference. The nucleotide sequence encoding the region of difference may readily be determined by one of ordinary skill in the art.

There are other methods for identifying the binding site of a drug to a protein. For example, the structure of the protein-candidate drug molecule complex, as well as the location of the binding site may be determined using techniques such as, for example, X-ray crystallography, electron microscopy or diffraction, nuclear magnetic resonance (NMR) spectroscopy, molecular modeling, and the like.

For example, Smith et al (Journal of Medicinal Chemistry (2003), 46(10), 1831-1844) utilized X-ray crystallography to provide information on the interactions between inhibitors and the HIV-1 enzyme. In another example, Pauly et al (Biochemistry 2003, 42, 3203-3213) utilized X-ray crystallography to provide information on the specificity determinants of human cathepsin S.

Alternatively, the amino acid sequence of the animal protein may be compared with the amino acid sequence of the human protein. The amino acid sequence may be determined by biochemical techniques, such as, for example peptide sequencing. Alternatively, the amino acid sequence may be obtained from databases, such as, for example, GenBank. A region on the animal protein that differs by at least one amino acid from the corresponding region on the human protein may be identified as a region of difference. There may be one, or more than one region of difference identified. The methods of the present invention employ a single zinc finger nuclease that is designed to bind to a specific target locus for a single region of difference. There may be a single region of difference identified, or there may be a plurality of regions identified. In the case of a plurality of regions identified, alteration of a single region of difference by the methods of the present invention may result in the desired effect.

In the case where alteration of a single region of difference does not achieve the desired effect, more than one, or all of the identified regions of differences may have to be altered. In the case where more than one region of difference is altered, sequential alteration of the identified regions may be achieved, employing a single zinc finger nucleases designed to bind to a specific target locus in sequential reactions for each single region of difference. Alternatively, more than one region of difference can be replaced at a time. In this case, a contiguous region including two or more regions of difference separated by regions of identity may be treated in these techniques as a single region of difference.

A target locus for the determined region of difference may be determined via computer analysis of the DNA sequence of the region of difference by a software program. Dobbs & Voytas Laboratories, College of Genetics, Development and Cell Biology, at the University of Iowa, disclose a computer program that may be suitable for use in the present invention (http://bindr.gdcb.iastate.edu/ZFPTFWeb). The design of a zinc finger nuclease for the calculated target locus may also be possible via computer software (http://www.scripps.edu/mb/barbas/zfdesign/zfdesignhome.php).

### Zinc Finger Nucleases

The present invention utilizes DNA double-stranded breaks to increase the efficiency of homologous recombination. In order to target homologous recombination at the region of difference, a target locus is identified and a specific zinc finger nuclease is generated that is capable of binding to the target locus and cleaving the DNA at the appropriate location to facilitate homologous recombination at the target locus.

A zinc finger nuclease of the present invention is a chimeric protein molecule capable of directing targeted genetic recombination or targeted mutation in a cell by causing a double-stranded break at a target locus. A zinc finger nuclease of the present invention comprises a DNA-binding domain and a DNA-cleavage domain separated by a linker peptide, wherein the DNA binding domain includes at least one zinc finger and is operatively linked to a DNA-cleavage domain by a linker peptide. In one embodiment, the zinc finger DNA-binding domain is at the N-terminus of the chimeric protein molecule and the DNA-cleavage domain is located at the C-terminus of the molecule. Examples of zinc finger nucleases suitable for use in the present invention are disclosed in WO 03/087341.

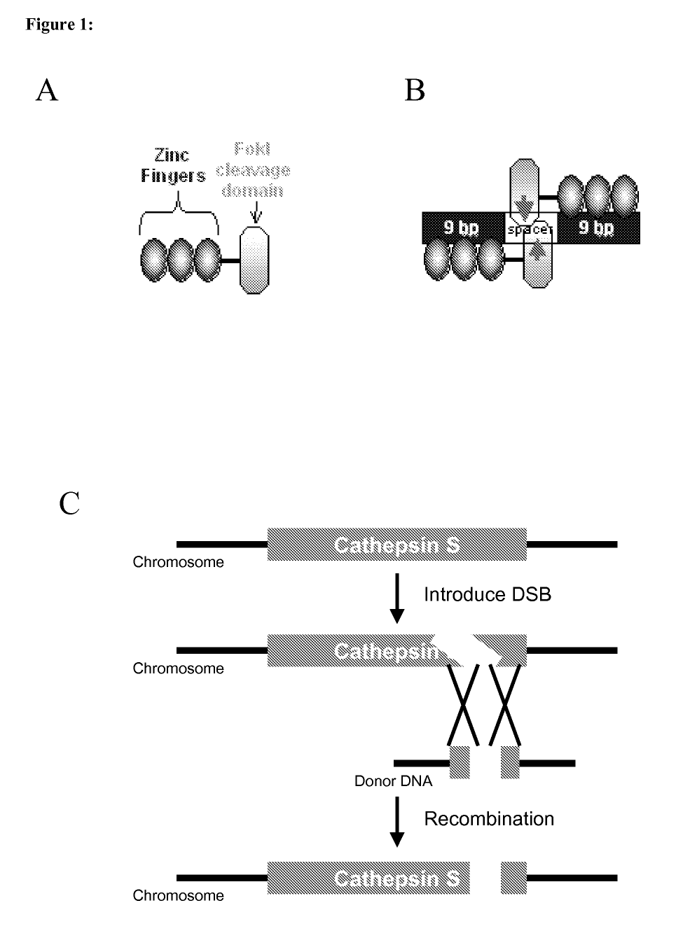

The specificity of the zinc finger nuclease for the target locus may by increased by increasing the number of zinc fingers in the DNA binding domain of the zinc finger nuclease. In one embodiment of the present invention, the DNA binding domain of the zinc finger nuclease contains one zinc finger. In an alternate embodiment, the DNA binding domain of the zinc finger nuclease contains three zinc fingers or alternatively a sufficient number of zinc fingers to facilitate homologous recombination at the desired location. In one embodiment, the zinc finger nuclease is a chimeric protein wherein a DNA binding domain comprising three Cys2His2 type zinc fingers, is operably linked to the non-specific DNA cleavage domain of the bacterial FokI restriction endonuclease (FIG. 1A). According to this embodiment, each zinc finger contacts three consecutive base pairs of DNA creating a nine base-pair recognition sequence for the zinc finger nuclease DNA binding domain.

The length of the linker peptide that operatively links the DNA binding domain with the DNA cleavage domain may be altered to achieve a DNA double-strand break at the appropriate location to facilitate homologous recombination at the region of difference.

In one embodiment, zinc finger nucleases bind as monomers to their target locus. Alternatively, zinc finger nucleases bind as dimers to their target locus with each monomer using its zinc finger domain to recognize a “half-site” (FIG. 1B). Dimerization of zinc finger nucleases is mediated by the FokI cleavage domain which cleaves within a five or six base pair “spacer” sequence that separates the two inverted “half sites” (FIG. 1B).

The DNA recognition and/or the binding specificity of a zinc finger nuclease may be altered in order to accomplish targeted homologous recombination at any chosen locus in cellular DNA. Such modification may be accomplished using known molecular biology and/or chemical synthesis techniques. Methods for selecting optimum subsequences from a target locus for targeting by a zinc finger protein may be found, for example, in WO 00/42219. Zinc finger nucleases comprising zinc fingers having a wide variety of DNA recognition and/or binding specificities are within the scope of the present invention.

The zinc finger nucleases suitable for use in the present invention may be constructed by a “modular assembly” method, wherein modules consisting of single zinc fingers that bind to random DNA sequences (obtained by selection from randomized libraries, by rational design, or from naturally occurring domains) are used to assemble domains composed of three or six fingers. These modular constructs may then be screened to select for those that bind specifically to the target locus. Examples of screening methods to select specific zinc finger nucleases are found in Lee, D-k et al, Current Topics in Medicinal Chemistry, Volume 3, Number 6, February 2003, pp. 645-657(13).

### Donor DNA Construction

Donor DNA may be in the form of an oligonucleotide or a plasmid. The donor DNA comprises the human region of interest, as identified above, to be inserted into the animal or cellular model DNA. Sequences homologous to the target locus are included in the DNA construct encoding the donor DNA, flanking the nucleotide sequence encoding the human region of difference to be integrated into the genome of the animal cell. Flanking, in this context, simply means that target homologous sequences are located both upstream (5′) and downstream (3′) of the nucleotide sequence to be inserted. These sequences correspond to sequences upstream and downstream of the target locus in the animal cell.

In one embodiment, the homologous flanking sequences are each about 50 nucleotides in length. In other embodiments, the homologous flanking sequences are anywhere from about 20 to about 50 nucleotides in length, including flanking sequences of about 20, about 30 or about 40 nucleotides in length.

The length of the region of homology in the sequences flanking the region of difference varies. It may be influenced by, for example, species, the length of the sequence to be inserted, the nucleotide composition of the DNA flanking the target locus. These factors may readily be determined by one of skill in the art.

DNA encoding an identifiable marker may also be included with the donor DNA construct. Such markers may include a gene or sequence whose presence or absence conveys a detectable phenotype to the animal cell. Various types of markers may include, but are not limited to, selection markers, screening markers and molecular markers.

Selection markers are usually genes that may be expressed to convey a phenotype that makes an organism resistant or susceptible to a specific set of environmental conditions. Screening markers may also convey a phenotype that is a readily observable and distinguishable trait, such as Green Fluorescent Protein (GFP), beta glucuronidase (GUS) or beta-galactosidase.

Once the donor DNA construct is designed and assembled, using ordinary skills in the art of molecular biology, the donor DNA construct is then introduced into the cell, thus permitting recombination between the cellular sequences and the construct. The presence of a selectable marker in the donor DNA may permit the selection of cells that contain the construct.

### Nucleic Acid Delivery

Nucleic acid delivery herein referred to as “transformation” may be carried out by a variety of known techniques that depend on the particular requirements of each cell or organism. Such techniques have been worked out for a number of organisms and cells, and may be adapted without undue experimentation to animal cells.

The following are some examples of delivery systems useful in practicing the present invention:

Liposomalformulations: In certain broad embodiments of the invention, the oligo- or polynucleotides and/or expression vectors containing zinc finger nucleases and, where appropriate, donor DNA, may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are cationic lipid-nucleic acid complexes, such as lipofectamine-nucleic acid complexes. Lipids suitable for use according to the present invention may be obtained from commercial sources, such as, for example, Lipofectamine™ 2000 Transfection Reagent, Invitrogen, Calif. Liposomes used according to the present invention may also be made by different methods and such methods are known in the art. It is understood that the size of the liposomes will vary depending on the method of synthesis.

Adenoviruses: Human adenoviruses are double-stranded DNA tumor viruses with genome sizes of approximately 36 Kilo bases. As a model system for eukaryotic gene expression, adenoviruses have been widely studied and well characterized, and they exhibit a broad host range in vitro and in vivo. In lytically infected cells, adenoviruses are capable of shutting off host protein synthesis, directing cellular machineries to synthesize large quantities of viral proteins, and producing copious amounts of virus.

Other Viral Vectors as Expression Constructs: Other viral vectors may be employed as expression constructs in the present invention. Vectors derived from, for example, vaccinia virus, adeno-associated virus (AAV), and herpes viruses may be employed. Defective hepatitis B viruses, may be used for transformation of host cells.

Non-viral Methods: Several non-viral methods are contemplated by the present invention for the transfer into a host cell of DNA constructs encoding zinc finger nucleases and, when appropriate, donor DNA. These include calcium phosphate precipitation, microinjection, and receptor-mediated transfection. These techniques are all well known to those in the art.

### Sources of Cells

The methods of the present invention may be applicable to a wide range of cell types and organisms, such as, for example, an oocyte, a gamete, a germline cell in culture or in an animal, a somatic cell in culture or in an animal, or a mammalian cell.

Donor DNA is inserted into specific regions of genes in animal cells by the methods of the present invention. The cells may be expanded in culture following modification and then employed in an in vitro assay. Alternatively, the cells may be used to generate a transgenic animal. The resulting transgenic animal may be employed as an animal model of disease, or it may be employed as a source of tissues or organs.

The present invention is further illustrated, but not limited by, the following examples.

### EXAMPLE 1

### Humanization of Cathepsin S

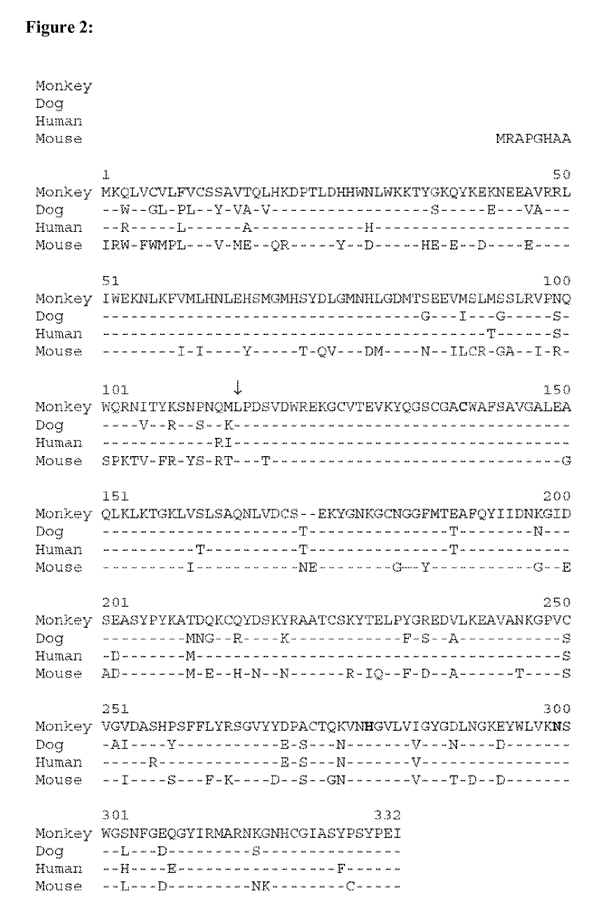

Determination of regions of differences between human and rat cathepsin S: The amino acid sequences of human, monkey, dog and mouse cathepsin S are compared to determine the number and location of any regions of difference. Human cathepsin S, monkey cathepsin S, dog cathepsin S and mouse cathepsin S cDNA sequences are aligned shown in FIG. 2. T188 in human cathepsin S is E188 in mouse cathepsin S.

Secondly, R256 in human cathepsin S is S256 in mouse cathepsin S, monkey and dog. The differences in candidate drug molecule binding affinities reported by Thurmond et al (JPET 308: 268-276, 2004) are attributed to the amino acid differences found.

Determination of Target Loci: Comparison of the mouse and human cathepsin S amino acid sequences reveals two regions of difference. Target loci for these regions of differences are determined using a software program developed by Dobbs & Voytas Laboratories, College of Genetics, Development and Cell Biology, at the University of Iowa (http://bindr.gdcb.iastate.edu/ZFPTFWeb).

Basic Options of the software: Sequence: Target loci for zinc finger arrays are identified in DNA sequences pasted into the window. Array sizes: If the ‘ZFN’ option is specified, the user can chose the number of zinc fingers (generally either three for four) in the left or right arrays. Note that each finger binds three nucleotides. If the ‘ZF Array’ option is specified (see below), the user can design arrays that vary from three to eight fingers in length. Spacer size: This parameter is only available in the ‘ZFN’ option. The user specifies the number of nucleotides between the zinc finger arrays. This distance is determined by the length of the amino acid linker between the zinc finger array and the nuclease domain. For standard linkers, the spacer is five or six base pairs, which provides the proper spacing between the zinc finger nuclease monomers so that they can interact to create a functional enzyme. ZFN/ZF array: The ‘ZFN’ option locates zinc finger nuclease target loci on both strands of the input DNA. The ‘ZF array’ option identifies target loci for zinc finger arrays that recognize one of the two DNA strands. Output from the ZF array option can be used to design zinc finger proteins for a variety of purposes, including use as transcriptional activators or repressors.

Induction of Targeted Mutations and Zinc Finger Design: Zinc finger nucleases are designed that specifically bind to, and induce double-stranded DNA breaks at the identified target loci in mouse cathepsin S.

The zinc finger nucleases are constructed by a “modular assembly” method, wherein modules consisting of single zinc fingers that bind to random DNA sequences (obtained by selection from randomized libraries, by rational design, or from naturally occurring domains) are used to assemble domains composed of three or six fingers. These modular constructs are then screened to select for those that bind specifically to the target loci.

Generation of the Donor DNA: In this example, two amino acids have been identified as being responsible for the differences in binding of the candidate drug molecules. Consequently, two donor DNA molecules are constructed, together with the necessary zinc finger nucleases to appropriately induce double-stranded DNA breaks and allow the specific incorporation of the donor DNA by homologous recombination. The donor DNA consists of an oligonucleotide comprising a sequence of nucleotides that enables the replacement of murine E 188 with T, flanked by nucleotides, whose sequence is homologous to the mouse gene. This oligonucleotide is inserted into a vector that allows the expression of the oligonucleotide in mammalian cells. A second donor DNA molecule includes a sequence of nucleotides enabling the replacement of murine S 256 with R, flanked by nucleotides, whose sequence is homologous to the mouse gene. This oligonucleotide is inserted into a vector that allows the expression of the oligonucleotide in mammalian cells. The donor DNA molecules also encode the genes for the appropriate zinc finger nucleases.

Generation of transgenic mice expressing “humanized” cathepsin S: Pronuclear DNA microinjection is the most commonly used method to generate transgenic mice. Fertilized oocytes are removed from the oviduct of a mouse, and the male pronucleus is microinjected with a solution containing the donor DNA. The injected eggs are cultured in vivo until the pronuclei have fused and the zygote has developed into a 2-cell embryo. The embryos are then transplanted into a surrogate mother, and pups are born 19-21 days later. Pups that developed from a zygote that successfully integrated the microinjected DNA have the donor DNA in every cell of their body. These heterozygous animals (called founders) can then be bred to obtain homozygous mice. Methods describing this procedure in detail may be found in “Microinjection and Transgenesis. Strategies and protocols. Springer laboratory manual. Editors: A. Cid-Arregui, A. Garcia-Carranca. Publisher: Springer-Verlag, Heidelberg, pp 275-284 (1998).

Publications cited throughout this document are hereby incorporated by reference in their entirety. Although the various aspects of the invention have been illustrated above by reference to examples and preferred embodiments, it will be appreciated that the scope of the invention is defined not by the foregoing description, but by the following claims properly construed under principles of patent law.

What is claimed is:

1) An animal model of disease, comprises at least one cell containing an endogenous protein that has been modified in at least one region to match its human homologue.

2) The animal model of claim 1, wherein endogenous protein is a drug target protein.

3) The animal model of claim 1, wherein the modification comprises an alteration in the binding affinity of the drug target protein to a candidate drug molecule.

4) The animal model of claim 2, wherein the drug target protein interacts with a second protein.

5) The animal model of claim 4, wherein the interaction of the drug target protein to the second protein is altered.

6) A method for generating an animal cell containing an endogenous protein that has been modified in at least one region to match its human homologue, comprising the steps of:
a) identifying at least one region in an animal protein that differs from a corresponding human protein homologue;
b) determining the nucleotide sequences that encodes the region of difference in the human and animal genes;
c) identifying a target locus for a zinc finger nuclease within the identified region of difference in the animal gene;
d) determining the nucleotide sequences that flank the region of difference in the animal gene;
e) generating donor DNA, comprising an oligonucleotide that encodes the human region of difference, flanked by the sequences determined in step d;
f) generating a zinc finger nuclease that is capable of binding to the target locus;
g) introducing the donor DNA and the zinc finger nuclease into animal cells, thereby inducing double-stranded breaks in the DNA of the animal cells,
h) allowing homologous recombination to occur between the animal DNA and the donor DNA, and;
i) selecting the animal cells where homologous recombination has occurred.

7) The method of claim 6, wherein the animal protein is a drug target protein.

8) The method of claim 7, wherein the binding affinity of the animal drug target protein to a candidate drug molecule is altered.

9) The method of claim 7, wherein the animal drug target protein interacts with a second protein.

10) The method of claim 9, wherein the interaction of the animal drug target protein and the second protein is altered.

11) Transgenic animals generated using a method comprising the method of claim 6.

12) A method for generating an animal protein that has been modified in at least one region to match its human homologue, comprising the steps of:
a) identifying at least one region in an animal protein that differs from a corresponding human protein homologue;
b) determining the nucleotide sequences that encodes the region of difference in the human and animal genes;
c) identifying a target locus for a zinc finger nuclease within the identified region of difference in the animal gene;
d) determining the nucleotide sequences that flank the region of difference in the animal gene;
e) generating donor DNA, comprising an oligonucleotide that encodes the human region of difference, flanked by the sequences determined in step d;
f) generating a zinc finger nuclease that is capable of binding to the target locus;
g) introducing the donor DNA and the zinc finger nuclease into animal cells, thereby inducing double-stranded breaks in the DNA of the animal cells,
h) allowing homologous recombination to occur between the animal DNA and the donor DNA;
i) selecting the animal cells where homologous recombination has occurred;
j) allowing the animal cells to express the modified protein, and;
k) isolating the purified protein.

13) The method of claim 12, wherein the animal protein is a drug target protein.

14) The method of claim 13, wherein the binding affinity of the animal drug target protein to a candidate drug molecule is altered.

15) The method of claim 13, wherein the animal drug target protein interacts with a second protein.

16) The method of claim 15, wherein the interaction of the animal drug target protein and the second protein is altered.
